(19) United States
(12) Patent Application Publication
(10) Pub. No.: US 2023/0285780 A1
(43) Pub. Date: Sep. 14, 2023

(54) MULTI-FUNCTION MASK

(71) Applicants: R. Nicholas Vincent Phillips-Reyes, Santa Rosa, CA (US); Sushanta Lokesh Parkih, Wernersville, PA (US)

(72) Inventors: R. Nicholas Vincent Phillips-Reyes, Santa Rosa, CA (US); Sushanta Lokesh Parkih, Wernersville, PA (US)

(21) Appl. No.: 17/692,021

(22) Filed: Mar. 10, 2022

(51) Int. Cl. A62B 18/00; A61M 11/04; A62B 23/02; A62B 18/10; A62B 9/00; A41D 13/11; B01D 46/42

(52) U.S. Cl. CPC A62B 18/003; A61M 11/04; A62B 23/02; A62B 18/10; A62B 9/006; A41D 13/11; B01D 46/4272

(57) ABSTRACT

Multi-function masks are presented including: a mask body forming a chamber, where the mask body includes an inner surface, an outer surface, an edge portion, a first intake portion, a second intake portion, a third intake portion, and an exhaust outlet portion, where the first intake portion passes filtered ambient air, where the second intake portion passes filtered and vaporized air, and where the third intake portion passes any of a fluid or a pressurized gas; a flexible contact portion positioned along the edge portion of the mask body for tightly contacting the mask body to a face when worn; and a filter unit positioned along the outer surface of the mask body for providing filtered ambient air to the first and second intake portions and for filtering exhaust air from the exhaust outlet portion.

### BACKGROUND

Currently, it is increasingly important for individuals to utilize personal protection equipment (PPE). For example, in a lumber processing setting, PPE might include a mask utilized to filter a user's breathable air from wood dust. Masks filtration characteristics may be selected depending on the specific environment in which the user is present. However, since masks are becoming more ubiquitous for use in personal, nonwork-related environments, additional functionality may be desirable. For example, a user having a respiratory illness may require supplemental oxygen. Presently, solutions for providing supplemental oxygen as well as protective air filtering are non-existent. Likewise for medicinal and recreational vapor use in combination with protective air filter and/or supplemental oxygen.

As such multi-function masks are presented herein.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented below.

As such, multi-function masks are presented including: a mask body forming a chamber, where the mask body includes an inner surface, an outer surface, an edge portion, a first intake portion, a second intake portion, a third intake portion, and an exhaust outlet portion, where the first intake portion passes filtered ambient air, where the second intake portion passes filtered and vaporized air, and where the third intake portion passes any of a fluid or a pressurized gas; a flexible contact portion positioned along the edge portion of the mask body for tightly contacting the mask body to a face when worn; a filter unit positioned along the outer surface of the mask body for providing filtered ambient air to the first and second intake portions and for filtering exhaust air from the exhaust outlet portion; and a user activated switch for closing the first intake such that intake air is switched to the second intake. In some embodiments, the first intake portion includes: an intake check valve for allowing passage of filtered ambient air toward the user and for preventing backflow of filtered ambient air. In some embodiments, the second intake portion includes: a vaporizer unit for providing vaporized air; and a one-way valve coupled with the vaporizer unit for allowing passage of vaporized air toward the user and for preventing backflow of vaporized air, where the user activated switch provides power to the vaporizer unit. In some embodiments, the second intake portion further includes: an indicator for indicating to the user that the second intake portion is engaged. In some embodiments, the indicator is selected from the group consisting of: a haptic motor indicator, an LED light indicator, and a sonic indicator. In some embodiments, the third intake portion includes: a through body fitting positioned along the outer surface, where the through body fitting is configured to receive any of at least a cap, an air-line, and a liquid line. In some embodiments, the air-line includes a check valve and an air-line tubing fitting each positioned outside the chamber. In some embodiments, the liquid line includes a liquid dry break check valve positioned outside the chamber and a bite valve positioned inside the chamber. In some embodiments, the exhaust outlet portion includes: an exhaust check valve for allowing passage of exhaust air through the mask body to the filter unit and for preventing backflow of exhaust air. In some embodiments, the user activated switch is configured to provide power to the vaporizer unit and to the indicator. In some embodiments, multi-function masks further include: an outer shell forming a protective cover over the filter unit, where the user activated switch is positioned along the outer shell. In some embodiments, the vaporizer unit is configured to deliver a substance selected from the group consisting of: herbal substances, pharmaceutical substances, vitamin substances, aromatherapy substances non-prescription substances, and therapeutic substances. In some embodiments, the outer shell further includes a removable shell cover, the removable shell cover providing an identifying indica of the user, where the removable shell cover is manufactured from a flexible fabric. In some embodiments, the filter unit includes: a filter support structure; and a filter, where the filter includes a single layer of air filtering media.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustrative representation of a multi-function mask system in accordance with embodiments of the present invention;

FIG. 2 is an illustrative representation of a multi-function mask in accordance with embodiments of the present invention;

FIG. 3 is an illustrative representation of an electronic vaporizer cartridge or vaporizer unit for use with a multi-function mask in accordance with embodiments of the present invention;

FIG. 4 is an illustrative exploded view representation of a multi-function mask in accordance with embodiments of the present invention;

FIG. 5 is an illustrative representation of a multi-function mask in accordance with embodiments of the present invention;

FIG. 6 is an illustrative representation of a through body fitting for a multi-function mask in accordance with embodiments of the present invention;

FIG. 7 is an illustrative representation of a multi-function mask in accordance with embodiments of the present invention;

FIG. 8 is an illustrative representation of a system for monitoring and controlling a multi-function mask in accordance with embodiments of the present invention; and

FIG. 9 is an illustrative exploded view representation of multi-function mask 900 in accordance with embodiments of the present invention.

### DETAILED DESCRIPTION

The present invention will now be described in detail with reference to a few embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

As will be appreciated by one skilled in the art, the present invention may be a system, a method, and/or a computer program product. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

A computer readable storage medium, as used herein, is not to be construed as being transitory signals/per se/, such as radio waves or other freely propagating electromagnetic waves, electromagnetic waves propagating through a waveguide or other transmission media (e.g., light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

In still other instances, specific numeric references such as “first material,” may be made. However, the specific numeric reference should not be interpreted as a literal sequential order but rather interpreted that the “first material” is different than a “second material.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

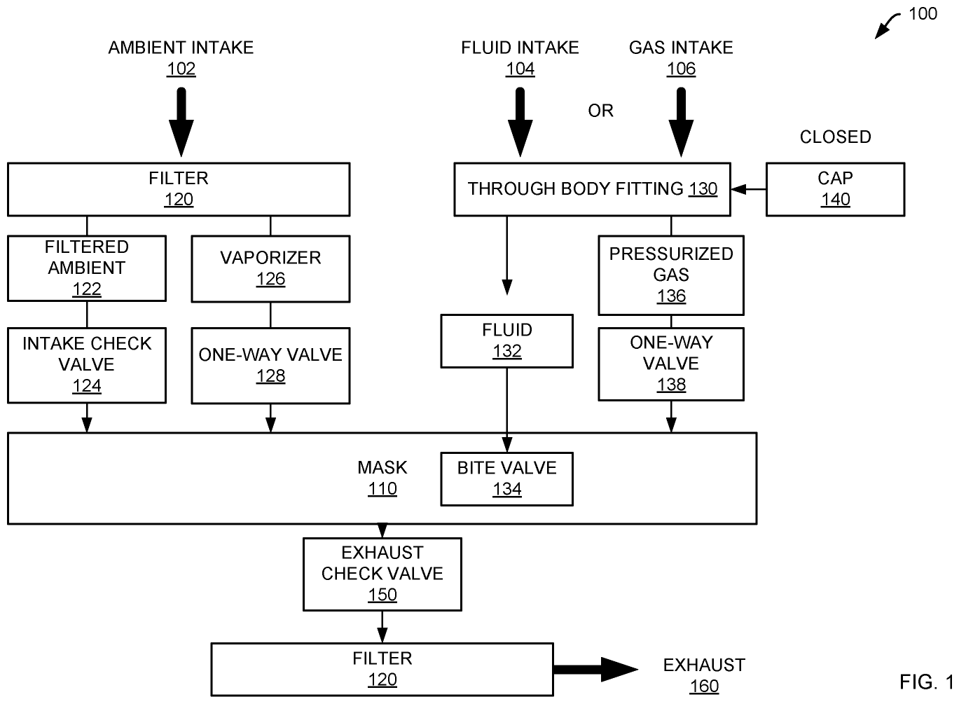

FIG. 1 is an illustrative representation of multi-function mask system 100 in accordance with embodiments of the present invention. Multi-function mask system embodiments provide a user with filtered air and any combination of various inhalants, and liquids. Multi-function mask systems filter both inlet and exhaust air from the user that provides safety for not only the user, but those sharing the user's space. Various inhalants and liquids provide both comfort and medications to a user while maintaining a safe breathing environment. As illustrated, multi-function mask system 100 includes ambient intake 102, fluid intake 104, and gas intake 106. Ambient intake refers to breathable air from the environment. Fluid intake refers to any fluid that the user selects for ready ingestion. Fluids may include water, juice, hydration liquids, medicinal formulations, or any other fluid or liquid without limitation. Gas intake refers to any gas that the user selects for ready inhalation. For example, a user requiring supplemental oxygen may utilize the multi-function mask to provide the oxygen required by a therapeutic regime. Multi-function mask embodiments provide a “multi-variable” drug delivery mask that has integrated functions to allow for the delivery of atomized (combusted) gases, compressed gases, and aerosol or sonicated gases without limitation.

Molecular mask embodiments include at least several operations as follows:

Ambient Air Filtration: In this operation, ambient intake 102 passes through filter 120 as filtered ambient air 122. The ambient air flows through intake check valve 124 into mask chamber 110 where the user inhales the filtered ambient air. Intake check valve embodiments serve to keep air moving in one direction thus preventing backflow. When air is expelled by the user, expelled air based through exhaust check valve 150 through filter 120 to exhaust 160.

Vaporizer: In this operation, ambient intake 102 passes through filter 120. A user activated switch on the front of mask simultaneously turns ON electronic vaporizer cartridge or vaporizer unit 126 by connecting the power with a momentary switch and closes the intake check valve 124 to redirect the incoming filtered air through the electronic vaporizer before passing through electronic vaporizer cartridge 126 input flow path. Vaporized air then passes though one-way valve 128 on the electronic vaporizer cartridge to mask chamber 110. The one-way valve prevents back flow of vaporized air. Vaporized air is then inhaled by the user. When the user activated switch is released, the electronic vaporizer cartridge turns OFF (i.e., the momentary switch releases and de-powers the electronic vaporizer cartridge) and the intake check valve is returned to the open position. In embodiments, the electronic vaporizer cartridge is a modular interchangeable electronic vaporizer that allows users to inhale vaporized substances while wearing a mask that also filters the incoming air. In embodiments, vaporized substances may include herbal substances, pharmaceutical substances, vitamin substances, aromatherapy substances, non-prescription substances, and therapeutic substances without limitation and without departing from embodiments provided herein.

Fluid Intake: In this operation, fluid intake 104 passes via through body fitting 130. Fluid may be provided via any container or vessel without limitation. In embodiments, the through body fitting is a multi-use port through which a variety of fluids or gases may be provided to a user. Through body fitting embodiments include a liquid dry break check valve that does not allow fluid or air to pass through when the liquid intake line disconnected. Fluid 132 delivery is controlled by bite valve 134, which is controlled by the user and is positioned in mask chamber 110. Bite valves are well-known in the art and may be provided in any configuration without departing from embodiments herein.

Gas Intake: In this operation gas intake 106 passes through thorough body fitting 130. Gases may be provided via any container or vessel without limitation. In embodiments, the through body fitting is a multi-use port through which a variety of fluids or gases may be provided to a user. Pressurized gas 136 passes through one-way valve 138 into mask chamber 110 whereupon the gas is inhaled by the user. In embodiments, this one-way valve is provided to prevent back pressure that allows the user to add the pressured gas to the normal filtered air. In this manner, multi-function mask systems provide an integrated “Direct Gas Delivery” feature inside of the mask where gaseous compounds flow through a mouthpiece, nasal cannulas, or nasal pillows directly to the user. Exhaled gas will pass through exhaust check valve 150 and through filter 120 as exhaust 160. In some embodiments, through body fitting may be closed with cap 140. In other embodiments, through body fitting may be closed in any manner known in the art without limitation.

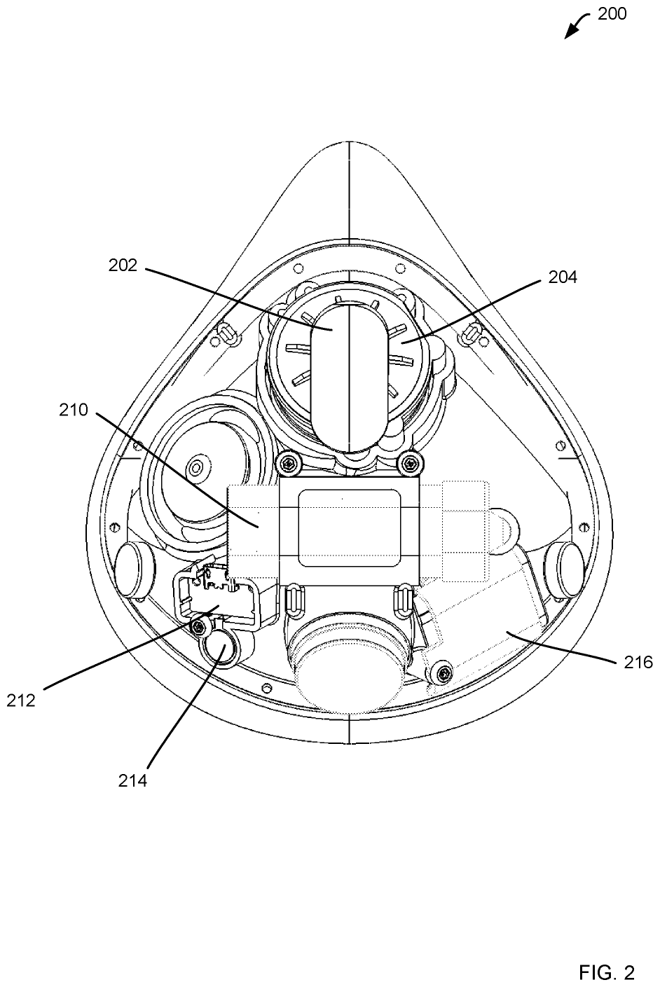

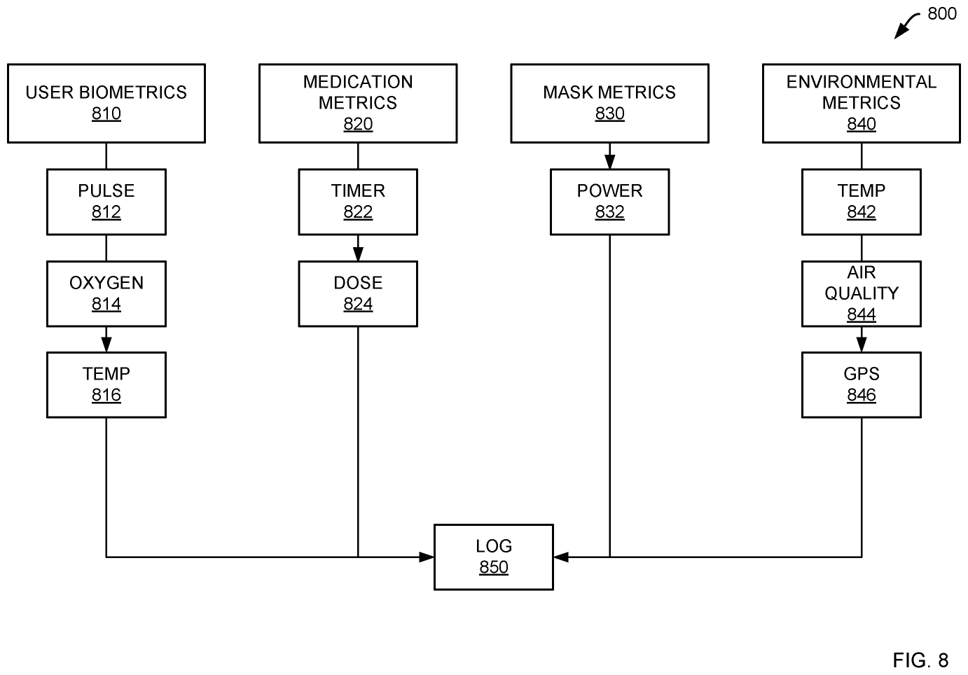

FIG. 2 is an illustrative representation of multi-function mask 200 in accordance with embodiments of the present invention. In particular, FIG. 2 illustrates components associated with an electronic vaporizer cartridge also referred to as a vaporizer unit embodiment. As illustrated multi-function mask includes user activated switch 202 that actuates both an electronic power switch to power vaporizer unit 210 and pad 204 to actuate the intake check valve. In operation, when the user activated switch is not activated, filtered ambient air flows through the ambient intake path to the user as disclosed above for FIG. 1. When the user activated switch is depressed, filtered ambient air flows through the vaporizer intake path to the user as disclosed above for FIG. 1. Further illustrated is haptic motor 214 that serves as a physical feedback indicator to the user that the vaporizer has been activated. Other indicator embodiments may include an LED light indicator or a sonic indicator without limitation. Use of an indicator is desirable so that a user can accurately monitor use and dose of vaporized air. In embodiments, the vaporizer is controlled by vaporizer PCB 212 and powered by power source 216. PCB functions will be described in further detail below for FIG. 8. In embodiments, the power source may be a battery, a rechargeable battery, or an external power source without limitation. Power may be controlled by an inhalation switch in embodiments.

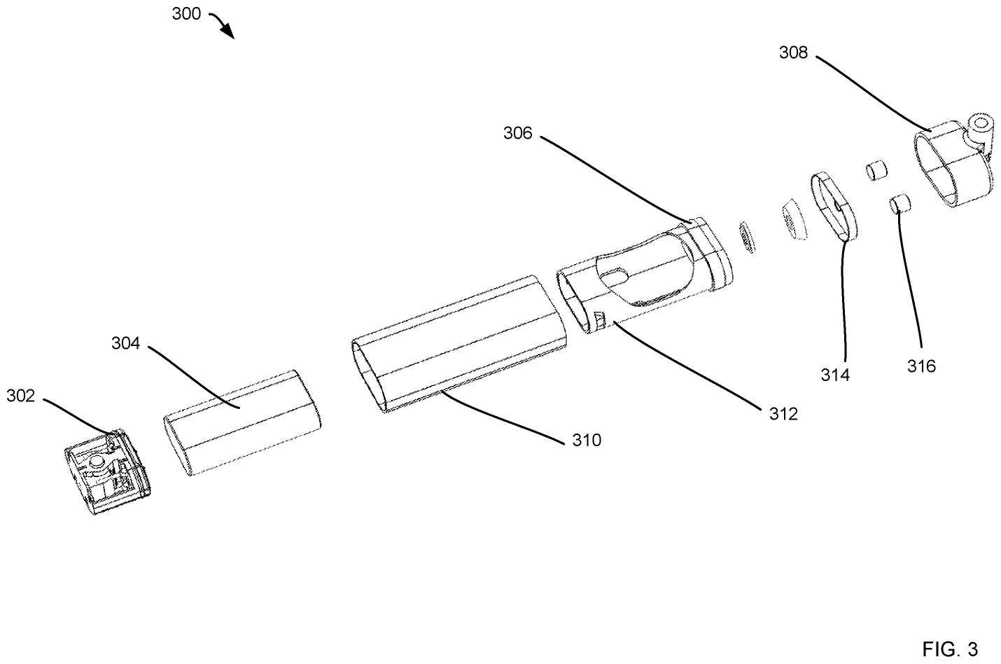

FIG. 3 is an illustrative representation of electronic vaporizer cartridge or vaporizer unit 300 for use with a multi-function mask in accordance with embodiments of the present invention. In order to facilitate use of a vaporizer, a mounting system has been devised to reduce the form factor of the vaporizer and well as to ease cartridge changes. As illustrated, vaporizer pod 304 is nested in pod cover 312 which is housed in cover 310. One-way valve 306 is integrated with pod cover 312. Removable cap 308 is coupled with pod cover 312 via magnets 316 and metal cover 314. Removable cap embodiments may be manufactured from any polymeric, semi-polymeric, metal, or metallic compound without limitation. Use of magnetic attachments provide easy access and cleaning so that a safe environment is maintained. Further illustrated, base 302 secures the vaporizer pod in the housing.

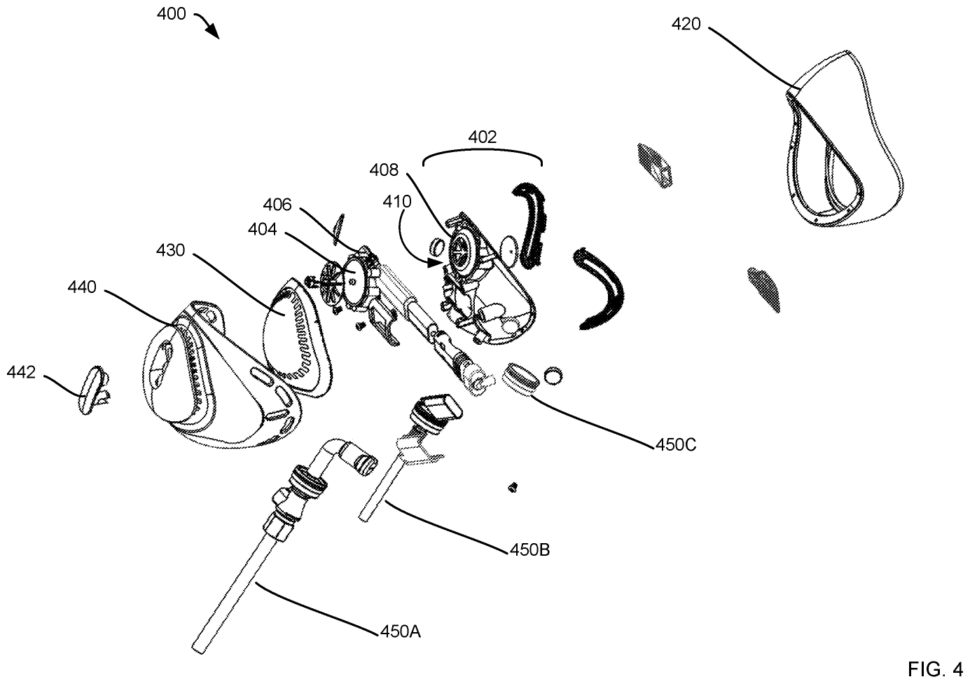

FIG. 4 is an illustrative exploded view representation of multi-function mask 400 in accordance with embodiments of the present invention. FIG. 4 is provided for clarity in understanding placement and location of the various mechanical structures. The indicated materials should not be construed as limiting with respect to embodiments provided herein, but rather as a guide as to the construction and utility of multi-function mask embodiments. As illustrated, multi-function mask 400 includes mask body 402 that forms a chamber over a user's mouth and nose. In embodiments, the mask body includes an inner surface, an outer surface, and an edge portion. Further, illustrated are ambient inlet 404, vapor inlet 406, through body port inlet 408, and exhaust 410. Flexible contact portion 420 is positioned along the edge portion of mask body 402 for tightly contacting the mask body to the face when worn. In embodiments, the flexible contact portion provides a seal for safety and efficacy. Filtration of ambient air is provided by filter unit 430 positioned along the outer surface of mask body 402. In embodiments, filters may be selected for any level of filtration without limitation. Filter unit embodiments filter ambient air that flows to ambient intake and vaporizer intake as disclosed above for FIG. 1. In addition, filter unit embodiments filter exhaust air thereby providing additional safe breathing environments for other users. Further illustrated is outer shell 440 that forms a protective cover over filter unit 430. As may be seen user activated switch 442 is positioned along the outer shell for ease of access. As noted above, user activated switches are configured to select between ambient intake and vaporizer intake as well as for powering vaporizer units. Still further illustrated are a variety of connections for through body fitting embodiments. For example, liquid line 450A may provide liquid to a user and includes a liquid dry break check valve positioned outside the mask body chamber and a bite valve positioned inside the mask body chamber. In addition, air-line 450B may provide pressurized gas to a user and includes a check valve and air-line tubing fitting positioned outside the mask body chamber. Finally, cap 450C is illustrated that serves to close the through body fitting.

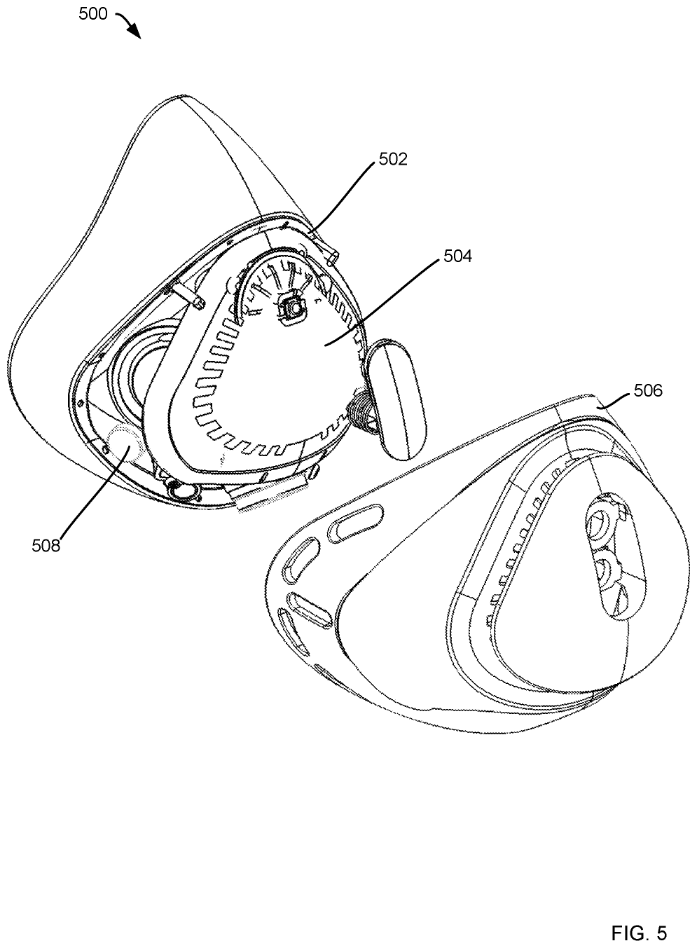

FIG. 5 is an illustrative representation of multi-function mask 500 in accordance with embodiments of the present invention. As illustrated, multi-function mask 500 includes mask body 502, which houses the structures required to provide the utility disclosed above. Further illustrated is removable filter unit 504 which filters both intake and exhaust air. Filters may be selected for any level of filtration without limitation. Further illustrated is outer shell 506, which may be attached with mask body 502 with magnets 508, with clips (not shown), or with any other suitable method known in the art without departing from embodiments provided herein. In addition, outer shell embodiments may include easily changed shell covers to enable different aesthetic or identifying looks.

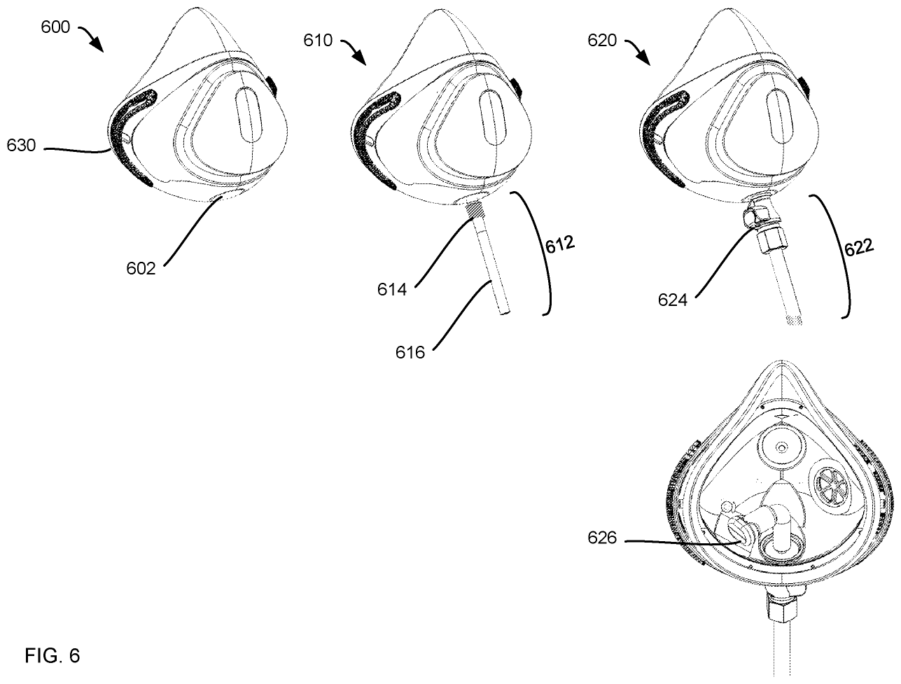

FIG. 6 is an illustrative representation of through body fittings for a multi-function mask in accordance with embodiments of the present invention. As noted above, in embodiments, the through body fitting is a multi-use port through which a variety of fluids and gases may be provided to a user. As illustrated, in a first example 600 through body fitting is sealed with cap 602. In a second example 610, through body fitting is illustrated having air-line 612 that includes check valve 614 and air-line tubing fitting 616. In a third example 620, through body fitting is illustrated having liquid line 622 that includes liquid dry break check valve 624 positioned outside of mask chamber and bite valve 626 positioned inside of mask chamber. Further illustrated are clips 630 that removably secure shell covers to enable different aesthetic or identifying looks.

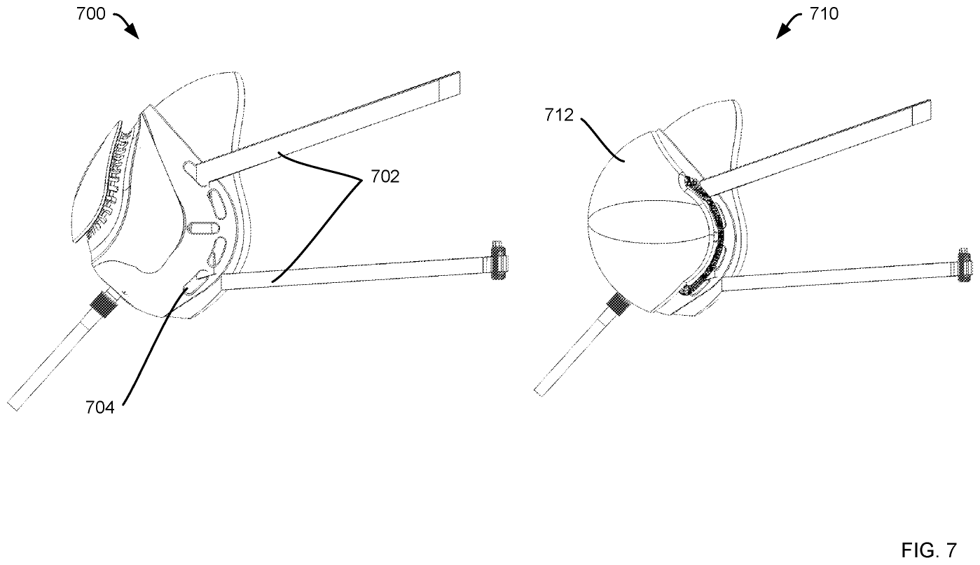

FIG. 7 is an illustrative representation of a multi-function mask 700 and 710 in accordance with embodiments of the present invention. As illustrated, straps 702 provide an aesthetic and/or functional feature of interchanging straps depending on user preference that can be removed and attached via the interchanging strap features 704 on the mask. In addition, shell cover 712 may enable different aesthetic or identifying looks. For example, a colored cover may be part of a uniform to readily identify or classify a group of users. Any indicia may be utilized with cover embodiments without departing from embodiments provided herein.

FIG. 8 is an illustrative representation of a system 800 for monitoring and controlling a multi-function mask in accordance with embodiments of the present invention. In embodiments, the various sensors and collected data may be organized to form part of an Internet of Things (IoT). That is, data may be shared among a user's selected entities so that cohesive data integration may be achieved. As such, several classes of data metrics may be gathered utilizing appropriate sensors. For example, user biometrics 810 may utilize sensors to gather user pulse data 812, user oxygen level data 814, and use temperature data 816 which may be logged 850 and, if desired, transmitted to a trusted entity. User biometrics may be useful to monitor the user's general health status when using the mask. Since the mask may provide vaporized products, pressurized gasses, and liquids, it may be useful to correlate dosage with relevant user biometrics. Additionally, medication metrics 820 may utilized sensors to monitor time data 822 and dose data 824 which may be logged 850 and, if desired, transmitted to a trusted entity. Dosage and dose administration may be useful to monitor the successful and consistent application of medications for the user. If a physician is a trusted entity, then the physician can monitor real-time therapeutic treatment. Further, mask metrics 830 may utilize sensors to gather power data 832 which may be logged 850 and, if desired, transmitted to a trusted entity. Power consumption modeling may be useful in determining how and when a multi-function mask embodiment requires maintenance and service. In critical therapy situations, a powered and functioning mask may prevent serious injury or death. Still further, environmental metrics 840 may utilized sensors to gather temperature data 842, air quality data 844, and global positioning system (GPS) data 832 which may be logged 850 and, if desired, transmitted to a trusted entity. External factors may, at times, affect a treatment regime. High temperature environments, for example, may necessitate an uptick in liquid intake. As such, utilizing environmental data, a treatment plan may be adjusted to deliver medications and fluids more effectively under varying environmental conditions.

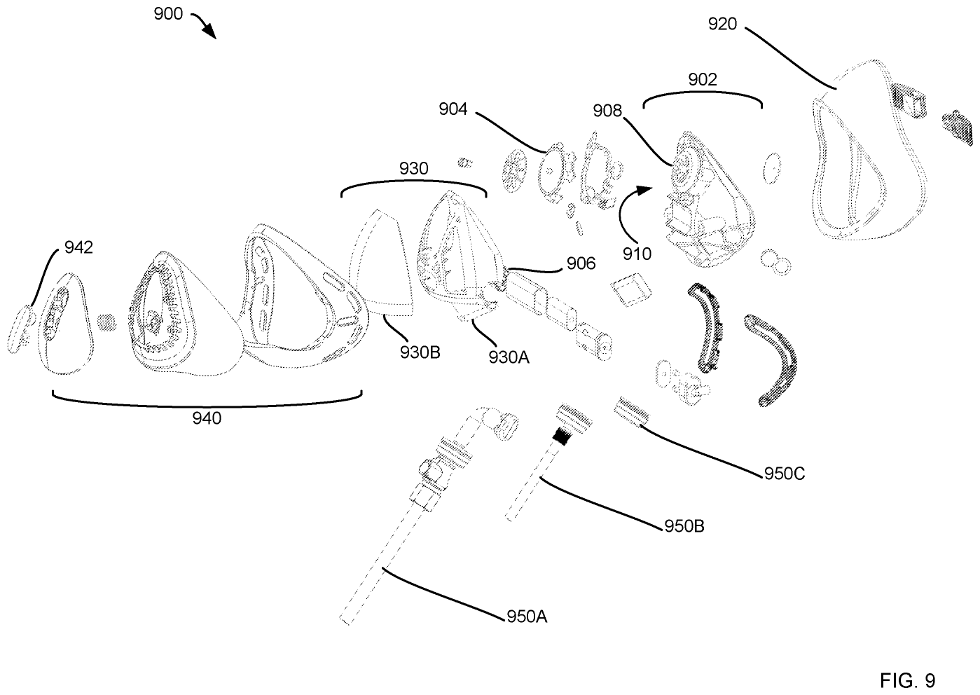

FIG. 9 is an illustrative exploded view representation of multi-function mask 900 in accordance with embodiments of the present invention. FIG. 9 is provided for clarity in understanding placement and location of the various mechanical structures. The indicated materials should not be construed as limiting with respect to embodiments provided herein, but rather as a guide as to the construction and utility of multi-function mask embodiments. As illustrated, multi-function mask 900 includes mask body 902 that forms a chamber over a user's mouth and nose. In embodiments, the mask body includes an inner surface, an outer surface, and an edge portion. Further, illustrated are ambient inlet 904, vapor inlet 906, through body port inlet 908, and exhaust 910. Flexible contact portion 920 is positioned along the edge portion of mask body 902 for tightly contacting the mask body to the face when worn. In embodiments, the flexible contact portion provides a seal for safety and efficacy. Filtration of ambient air is provided by filter unit 930 positioned along the outer surface of mask body 902. As illustrated, filter unit embodiments include a filter support structure 930A and filter 930B. Filter support structures embodiments include a battery cover and access for vaporizer units. In the illustrated embodiments, the filter is a single layer of air filtering media. In embodiments, filters may be selected for any level of filtration without limitation. Filter unit embodiments filter ambient air that flows to ambient intake and vaporizer intake as disclosed above for FIG. 1. In addition, filter unit embodiments filter exhaust air thereby providing additional safe breathing environments for other users. Further illustrated is outer shell 940 that forms a protective cover over filter unit 930. As may be seen user activated switch 942 is positioned along the outer shell for ease of access. As noted above, user activated switches are configured to select between ambient intake and vaporizer intake as well as for powering vaporizer units. Still further illustrated are a variety of connections for through body fitting embodiments. For example, liquid line 950A may provide liquid to a user and includes a liquid dry break check valve positioned outside the mask body chamber and a bite valve positioned inside the mask body chamber. In addition, air-line 950B may provide pressurized gas to a user and includes a check valve and air-line tubing fitting positioned outside the mask body chamber. Finally, cap 950C is illustrated that serves to close the through body fitting.

The terms “certain embodiments”, “an embodiment”, “embodiment”, “embodiments”, “the embodiment”, “the embodiments”, “one or more embodiments”, “some embodiments”, and “one embodiment” mean one or more (but not all) embodiments unless expressly specified otherwise. The terms “including”, “comprising”, “having” and variations thereof mean “including but not limited to”, unless expressly specified otherwise. The enumerated listing of items does not imply that any or all of the items are mutually exclusive, unless expressly specified otherwise. The terms “a”, “an” and “the” mean “one or more”, unless expressly specified otherwise.

While this invention has been described in terms of several embodiments, there are alterations, permutations, and equivalents, which fall within the scope of this invention. It should also be noted that there are many alternative ways of implementing the methods and apparatuses of the present invention. Furthermore, unless explicitly stated, any method embodiments described herein are not constrained to a particular order or sequence. Further, the Abstract is provided herein for convenience and should not be employed to construe or limit the overall invention, which is expressed in the claims. It is therefore intended that the following appended claims be interpreted as including all such alterations, permutations, and equivalents as fall within the true spirit and scope of the present invention.

What is claimed is:

1. A multi-function mask comprising:
a mask body forming a chamber, wherein
the mask body comprises an inner surface, an outer surface, an edge portion, a first intake portion, a second intake portion, a third intake portion, and an exhaust outlet portion, wherein
the first intake portion passes filtered ambient air, wherein
the second intake portion passes filtered and vaporized air, and wherein
the third intake portion passes any of a fluid or a pressurized gas;
a flexible contact portion positioned along the edge portion of the mask body for tightly contacting the mask body to a face when worn;
a filter unit positioned along the outer surface of the mask body for providing filtered ambient air to the first and second intake portions and for filtering exhaust air from the exhaust outlet portion; and
a user activated switch for closing the first intake such that intake air is switched to the second intake.

2. The multi-function mask of claim 1, wherein the first intake portion comprises:
an intake check valve for allowing passage of filtered ambient air toward the user and for preventing backflow of filtered ambient air.

3. The multi-function mask of claim 1, wherein the second intake portion comprises:
a vaporizer unit for providing vaporized air; and
a one-way valve coupled with the vaporizer unit for allowing passage of vaporized air toward the user and for preventing backflow of vaporized air, wherein the user activated switch provides power to the vaporizer unit.

4. The multi-function mask of claim 3, wherein the second intake portion further comprises:
an indicator for indicating to the user that the second intake portion is engaged.

5. The multi-function mask of claim 4, wherein the indicator is selected from the group consisting of: a haptic motor indicator, an LED light indicator, and a sonic indicator.

6. The multi-function mask of claim 1, wherein the third intake portion comprises:
a through body fitting positioned along the outer surface, wherein the through body fitting is configured to receive any of at least a cap, an air-line, and a liquid line.

7. The multi-function mask of claim 6, wherein the air-line comprises a check valve and an air-line tubing fitting each positioned outside the chamber.

8. The multi-function mask of claim 6, wherein the liquid line comprises a liquid dry break check valve positioned outside the chamber and a bite valve positioned inside the chamber.

9. The multi-function mask of claim 1, wherein the exhaust outlet portion comprises:
an exhaust check valve for allowing passage of exhaust air through the mask body to the filter unit and for preventing backflow of exhaust air.

10. The multi-function mask of claim 4, wherein the user activated switch is configured to provide power to the vaporizer unit and to the indicator.

11. The multi-function mask of claim 1, further comprising:
an outer shell forming a protective cover over the filter unit, wherein the user activated switch is positioned along the outer shell.

12. The multi-function mask of claim 3, wherein the vaporizer unit is configured to deliver a substance selected from the group consisting of: herbal substances, pharmaceutical substances, vitamin substances, aromatherapy substances non-prescription substances, and therapeutic substances.

13. The multifunction mask of claim 11, wherein the outer shell further comprises a removable shell cover, the removable shell cover providing an identifying indica of the user, wherein the removable shell cover is manufactured from a flexible fabric.

14. The multi-function mask of claim 1, wherein the filter unit comprises:
a filter support structure; and
a filter, wherein the filter comprises a single layer of air filtering media.
